(19) United States
(12) Patent Application Publication
(10) Pub. No.: US 2024/0199726 A1
(43) Pub. Date: Jun. 20, 2024

(54) ANTIBODIES TARGETING TALAROMYCES MARNEFFEI MP1P PROTEIN AND METHODS OF USE THEREOF

(71) Applicant: The University of Hong Kong, Hong Kong (CN)

(72) Inventors: Kwok Yung Yuen, Hong Kong (CN); Jianpiao Cai, Hong Kong (CN); Fuk Woo Jasper Chan, Hong Kong (CN); Chiu Yat Patrick Woo, Hong Kong (CN)

(21) Appl. No.: 17/930,919

(22) PCT Filed: Mar. 12, 2020
(86) PCT No.: PCT/CN2020/078978

§ 371 Date: Sep. 9, 2022

(51) Int. Cl. C07K 16/14; G01N 33/68

(52) U.S. Cl. CPC C07K 16/14; G01N 33/6854; C07K 2317/24; C07K 2317/565; G01N 2333/37; G01N 2800/26

(57) ABSTRACT

Provided are antibodies that specifically bind to a Talaromyces marneffei (T. marneffei) Mp1p protein. For example, such antibodies can be produced by a hybridoma cell line having the Patent Deposit Designation Number PTA-126527.

### BACKGROUND OF THE INVENTION

Talaromyces marneffei (T. marneffei) is a thermally dimorphic fungus which primarily affects subjects infected by the human immunodeficiency virus (HIV) or having acquired immunodeficiency syndrome (AIDS) and other immunocompromised subjects. The serological diagnosis of T. marneffei infection has been problematic as there are no reliable diagnostic assays available.

### BRIEF SUMMARY OF THE INVENTION

In one aspect, the disclosure provides an isolated antibody or antigen-binding portion thereof that specifically binds to a Talaromyces marneffei (T. marneffei) Mp1p protein and comprises:
a heavy chain variable region comprising a heavy chain CDR1, a heavy chain CDR2, and a heavy chain CDR3 (heavy chain CDR1-3), and
a light chain variable region comprising a light chain CDR1, a light chain CDR2, and a light chain CDR3 (light chain CDR1-3),
wherein the heavy chain CDR1-3 and light chain CDR1-3 have the same sequences as the corresponding CDRs of the antibody produced by a hybridoma cell line having the Patent Deposit Designation Number PTA-126527.

In some embodiments, the heavy chain variable region has the same sequence as that of the antibody produced by a hybridoma cell line having the Patent Deposit Designation Number PTA-126527.

In some embodiments, the light chain variable region has the same sequence as that of the antibody produced by a hybridoma cell line having the Patent Deposit Designation Number PTA-126527.

In some embodiments, the antibody is produced by a hybridoma cell line having the Patent Deposit Designation Number PTA-126527.

In some embodiments, the antibody is a monoclonal antibody. In some embodiments, the antibody is a chimeric antibody. In some embodiments, the antibody is a humanized antibody. In some embodiments, the antibody is an IgG1, IgG2, IgG3, or IgG4 antibody. In some embodiments, the antibody is a Fab, a F(ab′)₂, a scFv, or a bivalent scFv. In some embodiments, the antibody is conjugated to a detecting moiety.

In another aspect, the disclosure features a nucleic acid molecule encoding an isolated antibody described herein. The disclosure also features a vector comprising the nucleic acid molecule. The disclosure also features a host cell that expresses an isolated antibody described herein, wherein the host cell comprises a nucleic acid molecule described herein or a vector described herein, wherein the nucleic acid molecule or vector is expressed in the host cell. In some embodiments, the host cell is a hybridoma cell.

In another aspect, the disclosure features a hybridoma cell line having the Patent Deposit Designation Number PTA-126527.

In another aspect, the disclosure features a method for detecting the presence of a Talaromyces marneffei (T. marneffei) Mp1p protein in a sample, comprising contacting the sample with an antibody described herein, and detecting binding of the antibody to the T. marneffei Mp1p protein in the sample. In certain embodiments, the sample is a biological sample, for example, a biological sample isolated from a subject.

In some embodiments of the method, the subject is immunocompromised. In certain embodiments, the subject had been or is infected by the human immunodeficiency virus (HIV). In certain embodiments, the subject has acquired immunodeficiency syndrome (AIDS).

In some embodiments of the method, the method further comprises detecting binding of a secondary antibody to the antibody described herein. In certain embodiments, the secondary antibody is conjugated to a detecting moiety.

In some embodiments of the method, the presence of the T. marneffei Mp1p protein indicates a T. marneffei infection.

### BRIEF DESCRIPTION OF THE DRAWINGS

FIGS. 1A and 1B show the SDS-PAGE and Western blot analysis of the purified rMp1p protein.

FIGS. 2A-2C show the four antibody clones in Table 1 can bind to the Mp1p protein.

FIG. 2D shows the Western blot analysis of the four antibody clones in Table 1.

### DETAILED DESCRIPTION OF THE INVENTION

### I. Introduction

The present invention relates to the discovery of antibodies that bind to the Mp1p protein of Talaromyces marneffei (T. marneffei), especially the antibodies produced by a hybridoma cell line having the Patent Deposit Designation Number PTA-126527. As described further herein, the antibodies can be used in assays to detect the presence of T. marneffei Mp1p protein in a sample, which is an indication of a T. marneffei infection.

### II. Definitions

As used herein, the singular forms “a”, “an” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range indicate that the numeric value as well as reasonable deviations from the value known to the skilled person in the art, for example ±20%, ±10%, or ±5%, are within the intended meaning of the recited value.

As used herein, the term “antibody” refers to a protein functionally defined as a binding protein and structurally defined as comprising an amino acid sequence that is recognized by one of skill as being derived from a variable region of an immunoglobulin encoding gene. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity, including but not limited to Fab, F(ab′)₂, Fv, scFv, and bivalent scFv. An antibody can consist of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

The term “variable region” refers to a domain in an antibody heavy chain or light chain that derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 or CDR-H3 is located in the variable region of the heavy chain of the antibody in which it is found, whereas a VL CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and Contact CDR definitions.

The terms “antigen-binding portion” and “antigen-binding fragment” are used interchangeably herein and refer to one or more fragments of a molecule, e.g., an antibody, that retains the ability to specifically bind to an antigen (e.g., a T. marneffei Mp1p protein). Examples of antigen-binding fragments include, but are not limited to, a Fab fragment (a monovalent fragment consisting of the VL, VH, CL and CH1 domains), F(ab′)₂ fragment (a bivalent fragment comprising two Fab fragments linked by a disulfide bridge at the hinge region), single chain Fv (scFv), disulfide-linked Fv (dsFv), complementarity determining regions (CDRs), a VL (light chain variable region), a VH (heavy chain variable region), nanobodies, diabodies, each of which bind the antigen via a variable region, and other formats as described in Spiess et al., Mol. Immun. 67 (2015) 95-106, which is incorporated herein by reference, and any combination of these or any other functional portion of an immunoglobulin peptide capable of binding to a target antigen.

The term “epitope” refers to the area or region of an antigen to which a molecule, e.g., the CDRs of an antibody, specifically binds and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids (e.g., a linear epitope), or amino acids from different parts of the protein that are brought into proximity by protein folding (e.g., a discontinuous or conformational epitope).

The term “monoclonal antibody” refers to antibodies produced by a single clone of cells or a single cell line and consisting of or consisting essentially of antibody molecules that are identical in their primary amino acid sequence.

A “polyclonal antibody” refers to a pool of antibodies obtained from a heterogeneous population of antibodies in which different antibodies in the population bind to different epitopes of an antigen.

A “chimeric antibody” refers to an antibody molecule in which the constant region, or a portion thereof, is altered, replaced or exchanged so that the antigen-binding site (i.e., variable region, CDR, or portion thereof) is linked to a constant region of a different or altered class, effector function and/or species, or in which the variable region, or a portion thereof, is altered, replaced or exchanged with a variable region having a different or altered antigen specificity (e.g., CDR and framework regions from different species). In some embodiments, a chimeric antibody is a monoclonal antibody comprising a variable region from one source or species (e.g., mouse) and a constant region derived from a second source or species (e.g., human). Methods for producing chimeric antibodies are described in the art.

The term “humanized antibody” refers to an antibody that retains the reactivity of a non-human antibody while being less immunogenic in humans. This can be achieved, for instance, by retaining the non-human CDR regions and replacing the remaining parts of the antibody with their human counterparts. In some instances, it is necessary to retain particular non-human framework residues in order to retain the binding affinity and/or specificity of the non-human antibody once humanized.

The term “human antibody” or “fully human antibody” refers to an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “binding affinity” is used herein to refer to the strength of a non-covalent interaction between two molecules, e.g., between an antibody (or an antigen-binding portion thereof) and an antigen. Thus, for example, the term may refer to 1:1 interactions between an antibody (or an antigen-binding portion thereof) and an antigen, unless otherwise indicated. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between an antibody (or an antigen-binding portion thereof) and an antigen, but also apparent affinities for which K_{D}'s are calculated that may reflect avid binding.

The term “isolated,” as used with reference to a nucleic acid or protein (e.g., antibody), denotes that the nucleic acid or protein is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein (e.g., antibody) is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, 7-carboxyglutamate, and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

Naturally occurring amino acids are those encoded by the genetic code, as well as those amino acids that are later modified, e.g., hydroxyproline, 7-carboxyglutamate and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the subject, individual, or patient is a human.

### III. Antibodies

In one aspect, antibodies and antigen-binding portions of antibodies that specifically bind to the Mp1p protein of Talaromyces marneffei (T. marneffei) are provided. In some embodiments, the antibodies described herein are produced from a hybridoma cell line having the Patent Deposit Designation Number PTA-126527. In some embodiments, the antibodies described herein comprises: a heavy chain variable region comprising a heavy chain CDR1, a heavy chain CDR2, and a heavy chain CDR3 (heavy chain CDR1-3), and a light chain variable region comprising a light chain CDR1, a light chain CDR2, and a light chain CDR3 (light chain CDR1-3), in which the heavy chain CDR1-3 and light chain CDR1-3 have the same sequences as the corresponding CDRs of the antibody produced by a hybridoma cell line having the Patent Deposit Designation Number PTA-126527. In particular embodiments, the heavy chain variable region has the same sequence as that of the antibody produced by a hybridoma cell line having the Patent Deposit Designation Number PTA-126527 and/or the light chain variable region has the same sequence as that of the antibody produced by a hybridoma cell line having the Patent Deposit Designation Number PTA-126527.

The antibodies and antigen-binding portions of antibodies that specifically bind to the T. marneffei Mp1p protein can bind to an epitope within the Mp1p protein. In some embodiments, the epitope within the Mp1p protein can have at least 5 amino acids (e.g., at least 6, 7, 8, 9, 10, 12, 14, 16, 18, or 20 amino acids). In some embodiments, the epitope within the Mp1p protein can have between 5 and 20 amino acids (e.g., between 5 and 18, between 5 and 16, between 5 and 14, between 5 and 12, between 5 and 10, between 5 and 9, between 5 and 8, between 5 and 7, between 6 and 20, between 7 and 20, between 8 and 20, between 9 and 20, between 10 and 20, between 12 and 20, between 14 and 20, between 16 and 20, or between 18 and 20 amino acids). The Mp1p protein or an epitope within can include non-protein components, e.g., carbohydrates, as a result of glycosylation.

The antibody that specifically binds to the T. marneffei Mp1p protein can be a monoclonal antibody. The antibody can also be a chimeric antibody or a humanized antibody.

In some embodiments, the antibody can be conjugated to a detecting moiety. A detecting moiety can be any protein or organic small molecule that can provide a detectable signal either directly or indirectly. For example, a detecting moiety can be a fluorescent protein (e.g., green fluorescent protein (GFP), RFP, or YFP) or a small molecule fluorophore (e.g., fluorescein, rhodamine, Alexa Fluor). In other examples, a detecting moiety can be a biotin molecule, which can subsequently be detecting using a secondary binding molecule, such as a streptavidin molecule conjugated to a fluorescent protein or small molecule fluorophore or a streptavidin molecule conjugated to horseradish peroxide (HRP).

Methods for analyzing binding affinity, binding kinetics, and cross-reactivity of antibodies that bind to the T. marneffei Mp1p protein are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet™ (FortdBio, Inc., Menlo Park, CA)), and Western blot analysis.

### Preparation of Antibodies

For preparing antibodies, many techniques known in the art can be used. In some embodiments, antibodies are prepared by immunizing an animal or animals (e.g., mice, rabbits, or rats) with an antigen or a mixture of antigens for the induction of an antibody response. In some embodiments, the antigen used for raising the antibodies is the Mp1p protein from T. marneffei strain Tm-1 (Tm-1 Mp1p protein) or the Mp1p protein from T. marneffei strain Tm-4 (Tm-4 Mp1p protein), or a portion thereof. In particular embodiments, the antigen used for raising the antibodies is the Tm-1 Mp1p protein. In some embodiments, the antigen or mixture of antigens is administered in conjugation with an adjuvant (e.g., Freund's adjuvant). After an initial immunization, one or more subsequent booster injections of the antigen or antigens may be administered to improve antibody production. Following immunization, antigen-specific B cells are harvested, e.g., from the spleen and/or lymphoid tissue.

The antibodies produced can be purified using available techniques in the art, such as size-exclusion chromatography or affinity chromatography. The purity of the antibodies can be assessed using techniques such as sodium dodecyl sulfate-polyacrylamide gels (SDS-PAGE) and Western blotting. The binding affinity of purified antibodies to the Mp1p protein can be quantified by measuring an equilibrium dissociation constant (K_{D}). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform).

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Alternatively, phage or yeast display technology can be used to identify antibodies and Fab fragments that specifically bind to selected antigens. Techniques for the production of single chain antibodies or recombinant antibodies can also be adapted to produce antibodies. Antibodies can also be made bispecific, i.e., able to recognize two different antigens. Antibodies can also be heteroconjugates, e.g., two covalently joined antibodies, or immunotoxins.

Antibodies can be produced using any number of expression systems, including prokaryotic and eukaryotic expression systems. In some embodiments, the expression system is a mammalian cell expression, such as a hybridoma, or a CHO cell expression system. In particular embodiments, the antibodies described herein that bind to a T. marneffei Mp1p protein are produced from a hybridoma cell line having the Patent Deposit Designation Number PTA-126527. In embodiments in which an antibody comprises both a VH and VL region, the VH and VL regions may be expressed using a single vector, e.g., in a di-cistronic expression unit, or under the control of different promoters. In other embodiments, the VH and VL region may be expressed using separate vectors. Methods of generating and screening hybridoma cell lines, including the selection and immunization of suitable animals, the isolation and fusion of appropriate cells to create the hybridomas, the screening of hybridomas for the secretion of desired antibodies, and characterization of the antibodies are known to one of ordinary skill in the art.

### IV. Assays and Methods

The disclosure also provides assays using antibodies described herein (e.g., antibodies produced from a hybridoma cell line having the Patent Deposit Designation Number PTA-126527) to detect the presence of the T. marneffei Mp1p protein. In a first embodiment, the Mp1p protein of T. marneffei strain Tm-1 (Tm-1 Mp1p protein) is expressed from the yeast Pichia pastoris (P. pastoris). This P. pastoris expression system provides the necessary post-translational modifications, such as glycosylation, to produce a Tm-1 Mp1p protein that is structurally closer to its native form. Further, the Mp1p protein from T. marneffei strain Tm-1 (Tm-1 Mp1p protein) and the Mp1p protein from T. marneffei strain Tm-4 (Tm-4 Mp1p protein) are structurally different. The ligand-binding domain 1 (LBD1) of Tm-1 Mp1p protein and Tm-4 Mp1p protein are different, while the LBD2 of Tm-1 Mp1p protein and Tm-4 Mp1p protein are similar. The assays described herein use Tm-1 Mp1p protein.

Both mouse monoclonal antibodies and rabbit polyclonal antibodies against P. pastoris derived Tm-1 Mp1p protein were generated. These antibodies can be used as the coating antigen in an enzyme-linked immunosorbent assay (ELISA assay) to capture and bind to the Mp1p protein in a sample (e.g., a biological sample from a subject). A secondary antibody, such as a biotinylated antibody against the Mp1p protein, and an avidin conjugated to HRP can be used to provide a detectable signal.

In a second embodiment, the recombinant Tm-1 Mp1p protein produced from the P. pastoris expression system can be used as the coating antigen in an ELISA assay, which can then capture and bind to the antibodies against the Mp1p protein present in a sample (e.g., a biological sample from a subject). A secondary antibody, such as a goat anti-human IgG & IgM, can be used to provide a detectable signal.

The disclosure provides methods that detect the presence of a Talaromyces marneffei (T. marneffei) Mp1p protein in a sample by contacting the sample with an antibody described herein that binds to the T. marneffei Mp1p protein, and detecting binding of the antibody to the T. marneffei Mp1p protein in the sample. The sample can be a biological sample isolated from a subject suspected of having a T. marneffei infection. If the biological sample contains the Mp1p protein, it can be an indication that the subject had or has a T. marneffei infection.

The disclosure also provides methods that detect antibodies against the Mp1p protein present in a sample. As described herein, an Mp1p protein (e.g., a Tm-1 Mp1p protein produced from the P. pastoris expression system) can be used as the coating antigen in an ELISA assay. For example, the immobilized Tm-1 Mp1p protein can capture the anti-Mp1p antibodies if the antibodies are present in the sample. The sample can be a biological sample isolated from a subject suspected of having a T. marneffei infection. If the biological sample contains the anti-Mp1p antibodies, it can be an indication that the subject had or has a T. marneffei infection.

### V. Nucleic Acids and Vectors

In some embodiments, the antibodies as described herein are prepared using recombinant methods. Accordingly, in some aspects, the invention provides isolated nucleic acids comprising a nucleic acid sequence encoding any of the antibodies as described herein (e.g., an antibody produced from a hybridoma cell line having the Patent Deposit Designation Number PTA-126527). An isolated nucleic acid can also comprise a nucleic acid sequence encoding a CDR, a heavy chain variable region, or a light chain variable region described herein. Vectors comprising such nucleic acids are also provided.

In some embodiments, a polynucleotide (e.g., an isolated polynucleotide) comprises a nucleotide sequence encoding an antibody or antigen-binding portion thereof as described herein. In some embodiments, the polynucleotide comprises a nucleotide sequence encoding one or more amino acid sequences (e.g., CDR, heavy chain, light chain, and/or framework regions). In some embodiments, a polynucleotide is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

### VI. Examples

The present invention will be described in greater detail by way of specific examples. The following examples are offered for illustrative purposes only, and are not intended to limit the invention in any manner.

### Example 1. Production and Characterization of Recombinant Mp1p Protein of T. marneffei Strain Tm-1 (Tm-1 Mp1p Protein)

Inventors expressed the Mp1p gene encoding a secreted cell wall mannoprotein in T. marneffei strain Tm-1. The Mp1p protein from T. marneffei strain Tm-1 (Tm-1 Mp1p protein) and the Mp1p protein from T. marneffei strain Tm-4 (Tm-4 Mp1p protein) are structurally different. The ligand-binding domain 1 (LBD1) of Tm-1 Mp1p protein and Tm-4 Mp1p protein are different, while the LBD2 of Tm-1 Mp1p protein and Tm-4 Mp1p protein are similar. The N-terminal cleavable signal peptide and the C-terminal cleavable glycosylphosphatidylin-ositol (GPI) domain were removed from the full-length Mp1p gene. The gene was cloned into the P. pastoris expression vector pPIC9K (Invitrogen, Carlsbad, CA). The truncated Mp1p gene in P. pastoris strain GS115 (Invitrogen) was expressed and identified according to the manufacturer's instructions. A large scale expression of recombinant Mp1p protein (rMp1p) was optimized and the protein was purified by Ni-nitrilotriacetic acid affinity chromatography (Qiagen, Hilden, Germany).

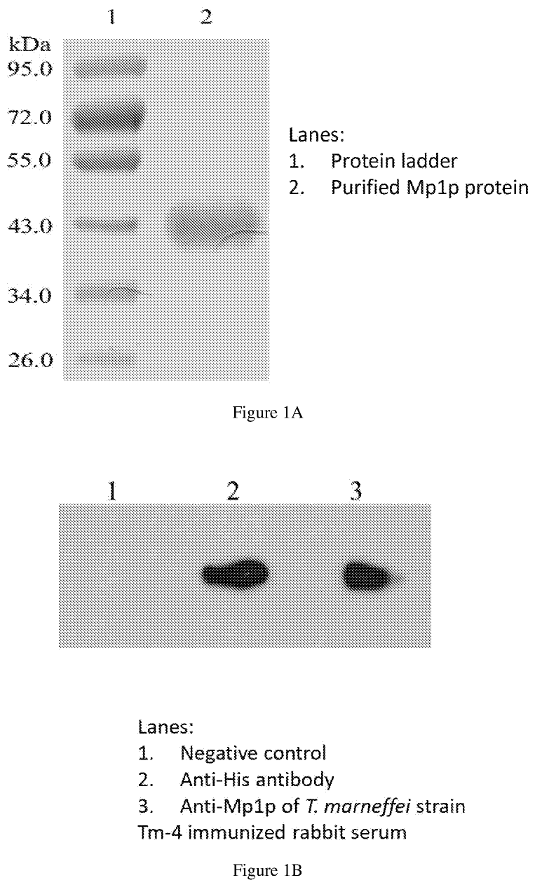

The purity of rMp1p was assessed by sodium dodecyl sulfate-polyacrylamide gels (SDS-PAGE) and Western blotting. In brief, the purified rMp1p was separated electrophoretically in a 12% gel and transferred to a nitrocellulose membrane. After blocking with 3% BSA and 7% skim milk (Sigma-Aldrich), the membrane was incubated with anti-serum from rabbit immunized with purified rMp1p from T. marneffei strain Tm-4 and anti-His monoclonal antibodies for 1 h at 37° C. After washing, the membrane was incubated with horseradish peroxidase (HRP; Sigma-Aldrich) conjugated goat anti-rabbit and goat anti-mouse antibody for 30 min at 37° C., and developed by incubation with Amersham ECL Advance Western Blotting Detection Kit (GE Healthcare, Fair-field, CT). The concentration of purified rMp1p was determined by using the Bicinchoninic Acid Protein Assay Kit (Sigma-Aldrich, St. Louis, MO) according to the manufacturer's instructions. FIGS. 1A and 1B show the SDS-PAGE and Western blot analysis of the purified rMp1p protein.

### Example 2. Characteristics of Monoclonal Antibodies Against Tm-1 Mp1p Protein

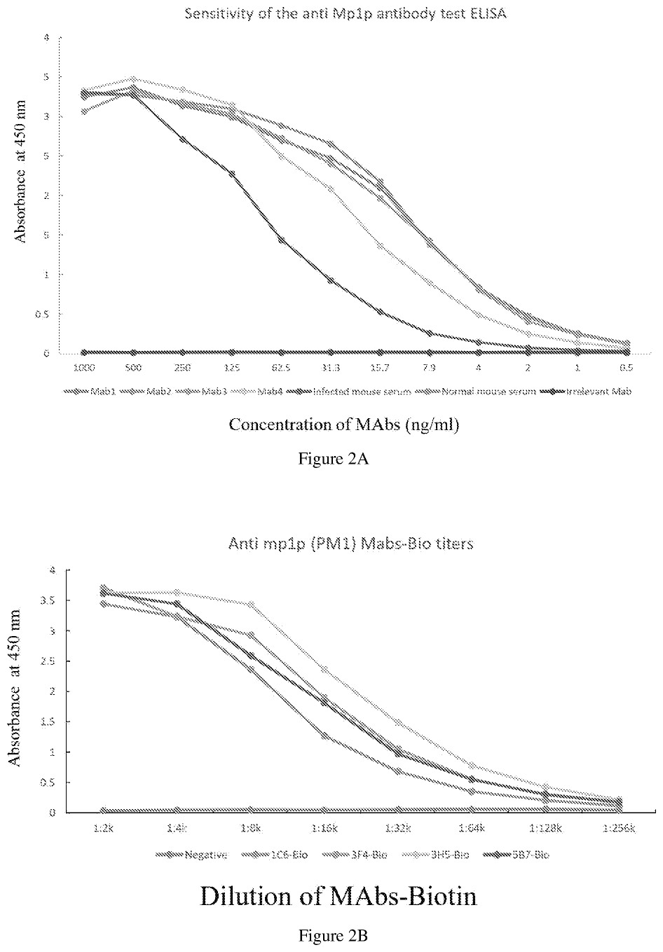

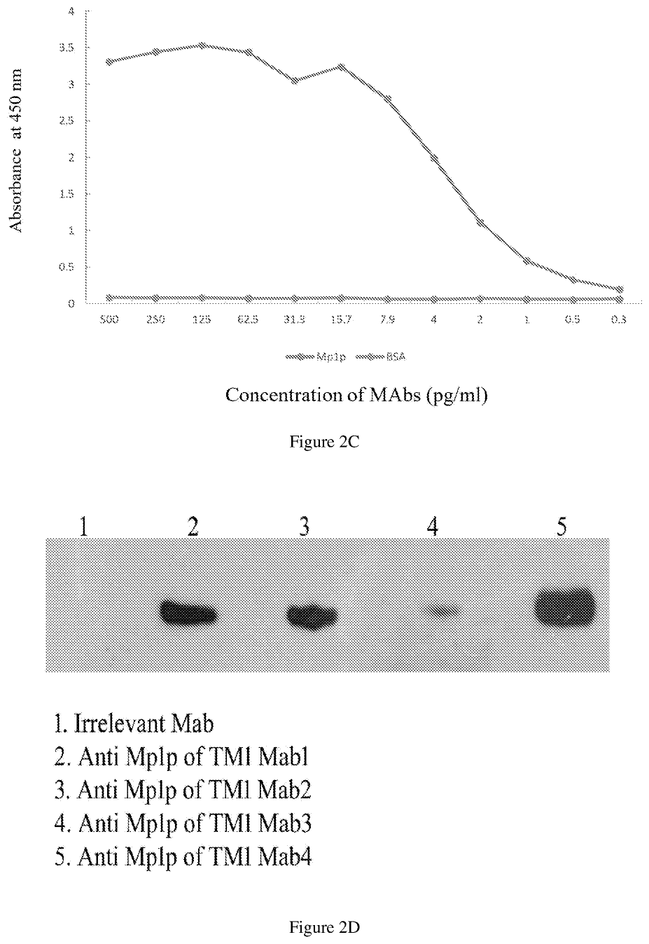

Inventors characterized the monoclonal antibodies by using Mouse MonoAb-ID Kie (ZYMED Laboratorise Inc, USA) according to the manufacturer's instructions with some modifications. Briefly, microwell plates were coated with 100 l/well of rMp1p protein (1 g/ml) overnight at 4° C. followed by incubation with a blocking reagent overnight at 4° C. After removal of the blocking solution, aliquots of 100 μl/well of monoclonal antibodies were added to microwell incubated at 37° C. for 1 h. After the plates were washed 6 times, 100 μl of diluted Goat Anti-Mouse IgGAM antibody was added to each well, and then the wells were incubated at 37° C. for 30 minutes. Then, they were washed 4 times with PBS-Tween and 100 μl of diluted HRP-Goat Anti-Rabbit IgG (H+L) was then added to each well and incubated at 37° C. for 30 minutes. Next, the wells were washed 4 times with PBS-Tween and then tetramethylbenzidine (TMB) substrate (100 μL/well) was added. The reaction was stopped after 10 min by the addition of 0.3N sulfuric acid, and the plates were then examined in an ELISA plate reader at 450 nm. Table 1 below shows the characteristics of four monoclonal antibodies produced from four different hybridoma cell strains. FIGS. 2A-2C further show the four antibody clones in Table 1 can bind to the Mp1p protein. FIG. 2D shows the Western blot analysis of the four antibody clones in Table 1.

TABLE 1

| | |
|---|---|
| Hybridoma | Characteristics with rMp1p |

| cell strain | Isotype | EIA | Western blot |
|---|---|---|---|
| 1C6 (Mab1) | IgG1 | +++ | ++ |
| 3F4 (Mab2) | IgG1 | +++ | ++ |
| 3H5 (Mab3) | IgG1 | +++ | + |
| 5B7 (Mab4) | IgG1 | +++ | +++ |

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

1. An isolated antibody or antigen-binding portion thereof that specifically binds to a Talaromyces marneffei (T. marneffei) Mp1p protein and comprises:
a heavy chain variable region comprising a heavy chain CDR1, a heavy chain CDR2, and a heavy chain CDR3 (heavy chain CDR1-3), and
a light chain variable region comprising a light chain CDR1, a light chain CDR2, and a light chain CDR3 (light chain CDR1-3),
wherein the heavy chain CDR1-3 and light chain CDR1-3 have the same sequences as the corresponding CDRs of the antibody produced by a hybridoma cell line having the Patent Deposit Designation Number PTA-126527.

2. The isolated antibody of claim 1, wherein the heavy chain variable region has the same sequence as that of the antibody produced by a hybridoma cell line having the Patent Deposit Designation Number PTA-126527.

3. The isolated antibody of claim 1, wherein the light chain variable region has the same sequence as that of the antibody produced by a hybridoma cell line having the Patent Deposit Designation Number PTA-126527.

4. The isolated antibody of claim 1, wherein the antibody is produced by a hybridoma cell line having the Patent Deposit Designation Number PTA-126527.

5. The isolated antibody of claim 1, wherein the antibody is a monoclonal antibody.

6. The isolated antibody of claim 1, wherein the antibody is a chimeric antibody.

7. The isolated antibody of claim 1, wherein the antibody is a humanized antibody.

8. The isolated antibody of claim 1, wherein the antibody is an IgG1, IgG2, IgG3, or IgG4 antibody.

9. The antigen-binding portion of the isolated antibody of claim 1, wherein the antigen-binding portion is a Fab, a F(ab′)2, a scFv, or a bivalent scFv.

10. The isolated antibody of claim 1, wherein the antibody is conjugated to a detecting moiety.

11. A nucleic acid molecule encoding an isolated antibody of claim 1.

12. A vector comprising the nucleic acid molecule of claim 11.

13. A host cell that expresses an antibody that specifically binds to a Talaromyces marneffei (T. marneffei) Mp1p protein,
wherein the antibody comprises:
a heavy chain variable region comprising a heavy chain CDR1, a heavy chain CDR2, and a heavy chain CDR3 (heavy chain CDR1-3); and
a light chain variable region comprising a light chain CDR1, a light chain CDR2, and a light chain CDR3 (light chain CDR1-3),
wherein the heavy chain CDR1-3 and light chain CDR1-3 have the same sequences as the corresponding CDRs of the antibody produced by a hybridoma cell line having the Patent Deposit Designation Number PTA-126527,
wherein the cell comprises a nucleic acid molecule or a vector encoding the antibody, and
wherein the antibody is expressed from the nucleic acid molecule or vector in the host cell.

14. The host cell of claim 13, wherein the host cell is a hybridoma cell.

15. A hybridoma cell having the Patent Deposit Designation Number PTA-126527.

16. A method for detecting the presence of a Talaromyces marneffei (T. marneffei) Mp1p protein in a sample, comprising contacting the sample with the antibody of claim 1, and detecting binding of the antibody to the T. marneffei Mp1p protein in the sample.

17. The method of claim 16, wherein the sample is a biological sample.

18. The method of claim 17, wherein the biological sample is isolated from a subject.

19. The method of claim 18, wherein the subject is immunocompromised.

20. The method of claim 18, wherein the subject had been or is infected by the human immunodeficiency virus (HIV), and/or wherein the subject has acquired immunodeficiency syndrome (AIDS).

21. (canceled)

22. The method of claim 16 further comprising detecting binding of a secondary antibody to the antibody, optionally wherein the secondary antibody is conjugated to a detecting moiety.

23. (canceled)

24. The method of claim 18, wherein detecting binding of the antibody to the T. marneffei Mp1p protein in the sample indicates a T. marneffei infection in the subject.
